(19) United States
(12) Patent Application Publication
(10) Pub. No.: US 2015/0209078 A1
(43) Pub. Date: Jul. 30, 2015

(54) VERESS NEEDLE

(71) Applicant: TEL-HASHOMER-MEDICAL RESEARCH, INFRASTRUCTURE AND SERVICES LTD., Ramat Gan (IL)

(72) Inventors: Avinoam Nevler, Reut (IL); Chaya Schwartz, Ramat Gan (IL); Nir Horesh, Ramat Gan (IL); Gil Har-Zahav, Ramat Gan (IL)

(73) Assignee: TEL-HASHOMER-MEDICAL RESEARCH, INFRASTRUCTURE AND SERVICES LTD., Ramat Gan (IL)

(21) Appl. No.: 14/421,170

(22) PCT Filed: Aug. 13, 2013
(86) PCT No.: PCT/US2013/054634

§ 371 Date: Feb. 12, 2015

Related U.S. Application Data
(60) Provisional application No. 61/682,321, filed on Aug. 13, 2012.

(51) Int. Cl. A61B 17/34; A61M 13/00; A61B 19/00

(52) U.S. Cl. CPC A61B 17/3474; A61B 17/3439; A61B 19/46; A61M 13/003; A61B 17/3415; A61B 2017/3441; A61B 2562/0247; A61B 2017/00867

(57) ABSTRACT

A Veress needle includes an outer needle having a shaft and a sharp distal point. The sharp distal point and a distal portion of the shaft are configured to penetrate tissue. A spring-loaded, inner cannula is disposed in the outer needle. The cannula has a dull tip and a gas exit aperture is formed near a distal end of the cannula. The outer needle has an outwardly expandable portion located on the distal portion of the shaft.

### FIELD OF THE INVENTION

The present invention relates generally to Veress needles, and particularly to a Veress needle with an expandable portion.

### BACKGROUND OF THE INVENTION

A typical Veress needle 1 of the prior art is shown in a simplified manner in FIGS. 1A-1B. Veress needle 1 has an outer needle 2 with a spring-loaded, inner cannula 3 (also referred to as a piston 3) disposed in the needle 2. The outer needle 2 has a sharp needle point 4 for cutting through tissue, such as abdominal wall tissue. The inner cannula 3 has a dull tip 5 to protect tissue from injury by the sharp, outer needle 2.

A gas exit aperture 6 is formed near the distal end of cannula 3. Aperture 6 fluidly communicates with a fluid source (not shown), such as carbon dioxide, via a connector 7 (e.g., a stopcock valve) typically located on a handle 8 to which the proximal end of needle 1 is assembled.

One widely-performed technique that uses the Veress needle is the so-called blind insertion, closed technique to gain access to a body space, such as the peritoneal space. In this technique, the Veress needle 1 is pushed against the tissue which one wishes to penetrate. Since the dull tip 5 extends distally further than needle point 4, the dull tip 5 first contacts the tissue. Pressing Veress needle 1 against the tissue causes the inner cannula 3 to retract proximally into the shaft of the outer needle 2. After needle 2 penetrates through the tissue into a body cavity, the inner cannula 3 springs distally forward, thereby exposing the gas exit aperture 6. Gas, such as carbon dioxide, is then passed through aperture 6 to inflate the space (e.g., the abdominal cavity), thereby creating a pneumoperitoneum. Afterwards, a trocar is inserted blindly into the newly created space for further surgical procedures. The gas provides a positive pressure which raises the inner body wall away from internal organs, thereby providing the surgeon with a region within which to operate and avoid unnecessary contact with the organs by the instruments inserted into the body cavity.

However, although the closed insertion technique is faster and easier than open techniques, insertion of the trocar after Veress needle penetration is associated with a higher rate of iatrogenic complications (injury to internal organs, major blood vessels, etc.), morbidity and even mortality.

### SUMMARY OF THE INVENTION

The present invention seeks to provide a novel Veress needle, as is described more in detail hereinbelow.

There is provided in accordance with an embodiment of the present invention a Veress needle including an outer needle having a shaft and a sharp distal point, wherein the sharp distal point and a distal portion of the shaft are configured to penetrate tissue, and a spring-loaded, inner cannula disposed in the outer needle, the cannula having a dull tip and a gas exit aperture formed near a distal end of the cannula, wherein the outer needle has an outwardly expandable portion located on the distal portion of the shaft. The expandable portion may be configured to expand radially outwards.

In accordance with an embodiment of the present invention the expandable portion includes deformable slats operatively linked to an actuator, wherein movement of the actuator causes the slats to deform and expand radially outwards.

In accordance with another embodiment of the present invention the expandable portion expands outwards by inflation.

In accordance with an embodiment of the present invention the expandable portion is constructed of a shape memory material.

In accordance with an embodiment of the present invention an inflatable member is disposed in the expandable portion.

In accordance with another embodiment of the present invention the outer needle and the inner cannula are disposed through a hollow portion of a trocar.

In accordance with an embodiment of the present invention the outer needle includes an external stopper positioned proximal to the expandable portion.

In accordance with an embodiment of the present invention the outer needle includes alignment structure positioned proximal to the expandable portion.

In accordance with an embodiment of the present invention a locking mechanism is operative to maintain the inner cannula in a distally extended state and prevent exposure of the sharp distal point.

### BRIEF DESCRIPTION OF THE DRAWINGS

The present invention will be understood and appreciated more fully from the following detailed description taken in conjunction with the drawings in which:

FIGS. 1A-1B are simplified illustrations of a Veress needle of the prior art;

FIG. 2 is a simplified pictorial illustration of a Veress needle, constructed and operative in accordance with a non-limiting embodiment of the present invention;

FIGS. 3A and 3B are simplified pictorial illustrations of the Veress needle after insertion through a tissue wall and expansion of an expandable portion of the outer needle, and respectively before and after pulling the Veress needle proximally against the inner tissue wall, in accordance with an embodiment of the present invention;

FIG. 4A is a simplified pictorial illustration of a Veress needle and trocar assembled together, in accordance with a non-limiting embodiment of the present invention; and

FIG. 4B is an enlarged illustration of the connection between the Veress needle and the trocar.

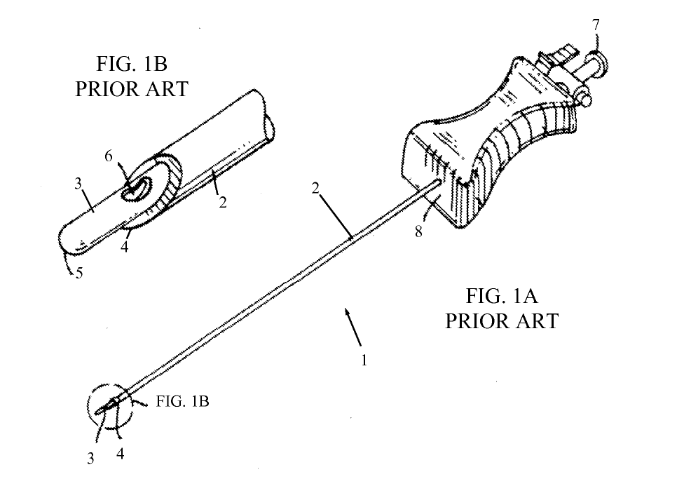

### DETAILED DESCRIPTION OF EMBODIMENTS

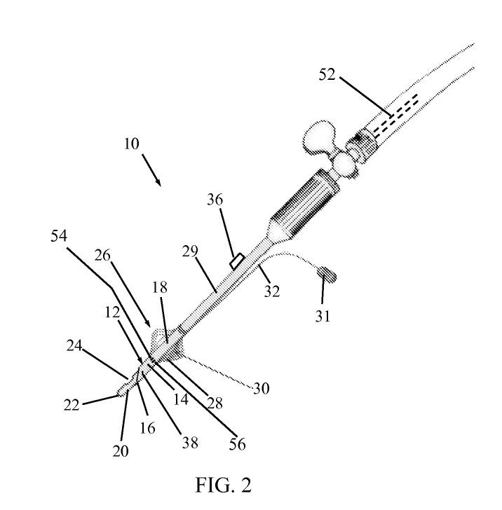

Reference is now made to FIG. 2, which illustrates a Veress needle 10, constructed and operative in accordance with a non-limiting embodiment of the present invention.

Veress needle 10 includes an outer needle 12 having a shaft 14 and a sharp distal point 16. The sharp distal point 16 and a distal portion 18 of shaft 14 are configured to penetrate tissue (not shown). A spring-loaded, inner cannula 20 (also referred to as a piston 20) is disposed in outer needle 12. Cannula 20 has a dull tip 22 and a gas exit aperture 24 formed near a distal end of cannula 20. Outer needle 12 has an outwardly expandable portion 26 located on the distal portion 18 of shaft 14. Expandable portion 26 may be configured to expand radially outwards.

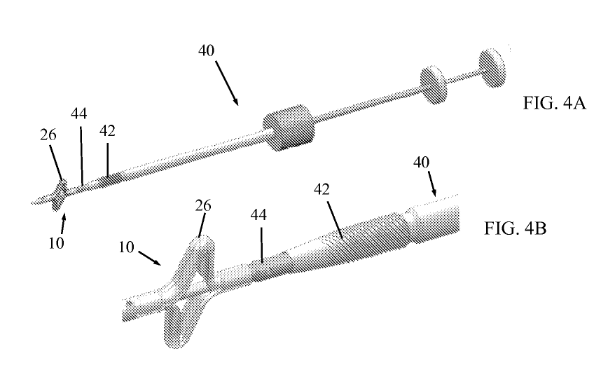

In the illustrated embodiment of FIG. 2, expandable portion 26 includes flexible, deformable slats 28. For example, a distal portion of slats 28 may be joined to shaft 14, whereas a proximal portion of slats 28 may extend from a tube 29 which slides over shaft 14. Slats 28 are initially flat and flush with the outer contour of shaft 14. Upon pushing the tube 29 towards distal point 16, the slats 28 buckle and deform radially outwards to the position seen in FIG. 2. Tube 29 thus serves as an actuator operatively linked to the slats 28. Alternatively, a handle or other kind of actuator may be provided for deforming the slats 28 to expand them radially outwards. For example, a screw-mechanism may be used to expand slats 28 outwards, similar to that of a molly bolt (an example of a screw-mechanism is described further below with reference to FIGS. 4A and 4B). The slats 28 may be contracted back to being flush with the outer contour of shaft 14 by reversing the movement of tube 29, to permit withdrawal of the Veress needle 10.

In accordance with an embodiment of the present invention an inflatable member 30, such as a balloon, may be disposed in expandable portion 26. The inflatable balloon 30 expands outwards by inflation, such as by means of an inflation fluid (e.g., air or water), from a fluid source 31, which flows through a suitable lumen 32 (e.g., a flexible tube or a lumen in the Veress needle). Balloon 30 may help prevent tissue from getting pinched between slats 28 upon contraction of slats 28.

Alternatively, the expandable portion 26 is just the inflatable balloon 30 without slats 28.

In accordance with another embodiment of the present invention, the expandable portion is constructed of a shape memory material, such as but not limited to, NITINOL. The expandable portion may be pre-cooled and then heated or warmed after penetration through the tissue, whereupon the expandable portion (such as the struts 28) expands outwards due to its shape memory properties (without need for pushing the proximal end of shaft 14 towards distal point 16).

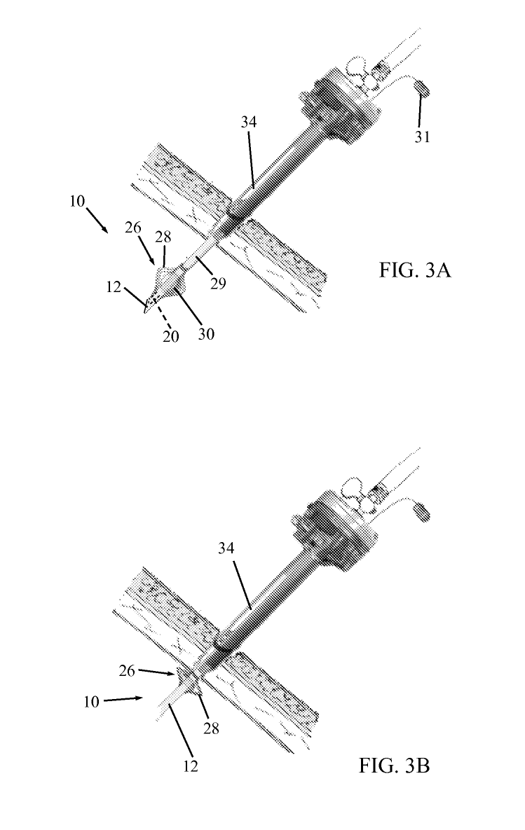

In use, the Veress needle 10 is inserted through tissue and the expandable portion 26 is expanded after insertion into the body (FIG. 3A). The expanded portion repels internal organs from the vicinity of needle 12 and provides a strong, sizable surface acting as a counter force when the Veress needle 10 is pulled back proximally against the tissue wall (FIG. 3B). This pulling force distances the tissue wall from near internal organs or vascular elements and increases safety for blind insertion of the trocar.

Referring to FIG. 3A, it is seen that outer needle 12 and inner cannula 20 may be disposed through a hollow portion of a trocar 34. In such an embodiment, the Veress needle 10 may be more elongate as to allow prior placement through trocar 34. In this embodiment, the Veress needle 10 serves as a guide for subsequent insertion of trocar 34, thus greatly increasing safety of the procedure. The trocar 34 is not inserted blindly; rather it is guided by the already placed Veress needle 10 and the expandable portion 26 blocks the sharp end of the trocar 34 from possibly damaging internal tissues.

Outer needle 12 may include an external stopper 36 (shown optionally in FIG. 2) positioned proximal to the expandable portion 26. Stopper 36 may help prevent inadvertent over-insertion of needle 12. Stopper 36 may be further shaped as alignment structure (by having, for example, a bubble vial) to better position or align the needle angle for perpendicular insertion into the body. Alternatively, outer needle 12 may include a dedicated alignment structure (again, such as a bubble vial) in addition to the stopper. The alignment structure may be between the trocar 34 and the outer needle 12.

In accordance with an embodiment of the present invention a locking mechanism 38 (such as a locking pin, seen in FIG. 2) is operative to maintain inner cannula 20 in a distally extended state and prevent exposure of the sharp distal point 16. For example, the user may insert the Veress needle 10 with the locking mechanism 38 set in a locked position that prevents the sharp distal needle point 16 from protruding outwards. The locking mechanism 38 is release to allow the needle to spring out only after the user feels the instrument has penetrated into the desired body cavity and is in a safe position with no danger of the needle puncturing sensitive tissue.

Optionally, as seen in broken lines in FIG. 2, an external probing element 52 can be inserted through the assembly (e.g., through shaft 14) to the inner cannula 20 to verify correct position of the needle and cannula. Additionally or alternatively, a pressure sensor 54 can be operatively connected to the needle 12 to verify correct position. Elevation of the abdominal wall causes a negative pressure inside the abdominal cavity, and this negative pressure can be sensed by pressure sensor 54.

Similarly, a force sensing element 56 (e.g., a strain gauge or load cell) that senses a change in tension can be used. The force sensing element 56 is operatively connected to the needle 12 to verify correct position. For example, force sensing element 56 can sense a loss of tensile resistance, which would indicate the cannula no longer blocks the needle and the needle can spring forward to puncture through the tissue wall. As another example, force sensing element 56 may be a syringe or other similar source of fluid pressure may be used to force gas or liquid through the needle. The amount of resistance to the fluid flow can indicate the position, that is, if the cannula blocks or does not block the needle; this pressure can be felt by the operator of the syringe. Alternatively, this pressure can be sensed by another force sensing element (e.g., a strain gauge or load cell). The force sensing element 56 may provide a visual, auditory and/or tactile indication to the user. The sensing may be continuous, periodic, or user dependent.

Reference is now made to FIGS. 4A and 4B, which illustrate Veress needle 10 and a trocar 40 assembled together, in accordance with a non-limiting embodiment of the present invention. An adapter 42 is used to connect between Veress needle 10 and trocar 40. The adapter 42 may be mounted by threaded engagement on a collar attachment 44 to the Veress needle 10. Adapter 42 may have a widened proximal end with a roughened surface (e.g., knurled surface) for easier grip. Threaded advancement of the adapter 42 can be used to achieve very fine controlled expansion of the expandable portion 26.

In summary, the invention may be used in a safe, controlled procedure as follows:

1. Manual elevation of the abdominal wall is performed.

2. The user punctures the tissue and verifies the position. Verification may be achieved by sensing a loss of resistance through the needle (The loss of resistance may be felt manually or sensed by using a pressure sensor). The loss of resistance may be felt or sensed in the movement or position of the piston, or in a syringe or guidewire probe used in the procedure. Other verification methods may use fluoroscopy or sonography.

3. The user may lock the internal piston (cannula) with the locking mechanism.

4. The user than deploys the expandable element and may continue to elevate the abdomen using the device

5. The user starts inflation.

6. The user advances the adapter to achieve controlled expansion of the opening in the abdominal wall.

7. The user advances the trocar safely

8. After the procedure, the user removes the Veress needle assembly.

A prototype system of the invention was tested over 20 times. The prototype system supported weights of 5.75 kgF up to the maximal strength of the tissue (6.75 KgF). No damage or mechanical malfunction was seen in any stage of the experiment.

Advantages of the invention include, without limitation:

a. Guided Insertion (one puncture vs. two)

b. Safe elevation of the abdominal wall

c. Rapid access to the surgical space, which allows fast “scope inside”—initial viewing, assessment and damage control

d. The stopper and/or locking mechanism that prevents uncontrolled entry into the surgical space

e. Controlled application of force

What is claimed is:

1. A Veress needle comprising:
an outer needle having a shaft and a sharp distal point, wherein said sharp distal point and a distal portion of said shaft are configured to penetrate tissue; and
a spring-loaded, inner cannula disposed in said outer needle, said cannula having a dull tip and a gas exit aperture formed near a distal end of said cannula,
wherein said outer needle has an outwardly expandable portion located on said distal portion of said shaft.

2. The Veress needle according to claim 1, wherein said expandable portion is configured to expand radially outwards.

3. The Veress needle according to claim 1, wherein said expandable portion comprises deformable slats operatively linked to an actuator, wherein movement of said actuator causes said slats to deform and expand radially outwards.

4. The Veress needle according to claim 1, wherein said expandable portion expands outwards by inflation.

5. The Veress needle according to claim 1, wherein said expandable portion is constructed of a shape memory material.

6. The Veress needle according to claim 1, further comprising an inflatable member disposed in said expandable portion.

7. The Veress needle according to claim 1, wherein said outer needle and said inner cannula are disposed through a hollow portion of a trocar.

8. The Veress needle according to claim 1, wherein said outer needle comprises an external stopper positioned proximal to said expandable portion.

9. The Veress needle according to claim 8, wherein said outer needle comprises alignment structure positioned proximal to said expandable portion.

10. The Veress needle according to claim 1, further comprising a locking mechanism operative to maintain said inner cannula in a distally extended state and prevent exposure of said sharp distal point.

11. The Veress needle according to claim 1, further comprising an external probing element inserted to said inner cannula.

12. The Veress needle according to claim 1, further comprising a pressure sensor operatively connected to said needle.

13. The Veress needle according to claim 1, further comprising a force sensing element operatively connected to said needle.
